(19) United States
(12) Patent Application Publication
(10) Pub. No.: US 2025/0135229 A1
(43) Pub. Date: May 1, 2025

(54) METHOD AND APPARATUS FOR RECONSTRUCTING DOSE DISTRIBUTION

(71) Applicant: Shanghai United Imaging Healthcare Co., Ltd., Shanghai (CN)

(72) Inventors: Mingming Zhang, Shanghai (CN); Fei Zhao, Shanghai (CN); Libin Wen, Shanghai (CN); Yanfang Liu, Shanghai (CN); Zhao Jin, Shanghai (CN); Fuwei Zhao, Shanghai (CN)

(21) Appl. No.: 18/934,309

(22) Filed: Nov. 1, 2024

(30) Foreign Application Priority Data
Nov. 1, 2023 (CN) 202311448317.7

(51) Int. Cl. A61N 5/10; G16H 20/40

(52) U.S. Cl. CPC A61N 5/1031; A61N 2005/1034; G16H 20/40

(57) ABSTRACT

A method for reconstructing a dose distribution for a region of interest includes determining a predicted source intensity distribution of a radiation source; determining a calculated transmission dose distribution based on the predicted source intensity distribution; determining a target source intensity distribution based on the calculated transmission dose distribution and the predicted source intensity distribution; and reconstructing the dose distribution of the region of interest based on the target source intensity distribution.

### CROSS-REFERENCE TO RELATED APPLICATIONS

This application claims priority to Chinese patent application No. 202311448317.7, entitled “Method and Apparatus for Reconstructing Dose Distribution, and Storage Medium”, filed on Nov. 1, 2023, which is hereby incorporated by reference in its entirety.

### TECHNICAL FIELD

The present application relates to the field of radiation delivery technologies, and in particular, to a method and apparatus for reconstructing a dose distribution, and a storage medium.

### BACKGROUND

During radiation delivery (for example, radiotherapy, radiation processing, radiation inspection, radiation testing, or the like), a radiation dose distribution is required to be determined to adjust a radiation delivery plan or monitor a completion degree of the radiation delivery.

For example, in-vivo three-dimensional dose monitoring is an important link of a radiotherapy process, and whether the dose distribution in a patient is reasonable can be determined by monitoring an in-vivo three-dimensional dose distribution in a radiation process.

In the related art, generally, a measured transmission dose of a dose monitoring device (for example, an electronic portal imaging device (EPID), a dose detector, or the like) is compared with a theoretical transmission dose to judge whether an actual radiation dose and a theoretical radiation dose of a region of interest are consistent, and a volume dose distribution is determined according to the judgment result.

However, the method in the related art cannot accurately acquire the volume dose distribution of the region of interest.

### SUMMARY

In a first aspect, the present disclosure provides a method for reconstructing a dose distribution for a region of interest, including determining a predicted source intensity distribution of a radiation source, determining a calculated transmission image based on the predicted source intensity distribution, determining a target source intensity distribution based on the calculated transmission image and the predicted source intensity distribution, and reconstructing the dose distribution of the region of interest based on the target source intensity distribution.

In some embodiments, determining the predicted source intensity distribution of the radiation source includes determining the predicted source intensity distribution of the radiation source based on a measured transmission image.

In some embodiments, determining the target source intensity distribution based on the calculated transmission image and the predicted source intensity distribution includes correcting the predicted source intensity distribution to obtain the target source intensity distribution.

In some embodiments, correcting the predicted source intensity distribution to obtain the target source intensity distribution includes determining whether the calculated transmission image meets a preset condition, and in the case that the calculated transmission image does not meet the preset condition, correcting the predicted source intensity distribution, such that the calculated transmission image corresponding to the corrected predicted source intensity distribution meets the preset condition, and determining the corrected predicted source intensity distribution corresponding to the calculated transmission image meeting the preset condition as the target source intensity distribution.

In some embodiments, the correction is performed by a machine learning-based correction model.

In some embodiments, determining the predicted source intensity distribution of the radiation source includes determining the predicted source intensity distribution of the radiation source based on a measured transmission image. Determining whether the calculated transmission image meets the preset condition includes determining a difference between the measured transmission image and the calculated transmission image, and determining whether the calculated transmission image meets the preset condition according to the difference.

In some embodiments, the difference includes a difference matrix, and elements of the difference matrix include gray differences between corresponding pixels of the measured transmission image and the calculated transmission image. Determining whether the calculated transmission image meets the preset condition according to the difference includes, if the difference matrix is smaller than or equal to a preset difference matrix, determining that the calculated transmission image meets the preset condition, or otherwise, determining that the calculated transmission image does not meet the preset condition.

In some embodiments, the method further includes training the correction model, and training the correction model includes training an initial correction model using predicted source intensity distribution samples to obtain the correction model.

In some embodiments, determining the calculated transmission image based on the predicted source intensity distribution includes determining the calculated transmission image using a dose algorithm based on the predicted source intensity distribution, and the dose algorithm includes a Monte Carlo dose algorithm.

In some embodiments, the target source intensity distribution includes a plurality of target source intensity distributions corresponding to different radiation angles of the radiation source respectively. Reconstructing the dose distribution of the region of interest based on the target source intensity distribution includes obtaining a plurality of dose distributions of the region of interest corresponding to the plurality of target source intensity distributions respectively and combining the plurality of dose distributions to obtain a combined dose distribution of the region of interest.

In some embodiments, the target source intensity distribution includes a plurality of target source intensity distributions corresponding to a same radiation angle of the radiation source respectively. Reconstructing the dose distribution of the region of interest based on the target source intensity distribution includes combining the plurality of target source intensity distributions to obtain a combined target source intensity distribution, and obtaining the dose distribution of the region of interest based on the combined target source intensity distribution.

In some embodiments, the region of interest includes a phantom or a specified part of the phantom, and the method further includes comparing the reconstructed dose distribution of the region of interest with a planned dose distribution.

In a second aspect, the present disclosure provides a method for reconstructing a dose distribution for a region of interest, including determining a predicted source intensity distribution of a radiation source, determining a calculated transmission dose distribution based on the predicted source intensity distribution, determining a target source intensity distribution based on the calculated transmission dose distribution and the predicted source intensity distribution, and reconstructing the dose distribution of the region of interest based on the target source intensity distribution. Determining the target source intensity distribution based on the calculated transmission dose distribution and the predicted source intensity distribution includes determining whether the calculated transmission image meets a preset condition, and in the case that the calculated transmission image does not meet the preset condition, correcting the predicted source intensity distribution, such that the calculated transmission image corresponding to the corrected predicted source intensity distribution meets the preset condition, and determining the corrected predicted source intensity distribution corresponding to the calculated transmission image meeting the preset condition as the target source intensity distribution.

In a third aspect, the present disclosure provides a non-transitory computer-readable storage medium having a computer program stored thereon. The computer program, when executed by a processor, causes the processor to perform the method for reconstructing a dose distribution for a region of interest according to any one of the embodiments described above.

In a fourth aspect, the present disclosure provides a computer device, which includes a memory storing a computer program and a processor. The processor, when executing the computer program, is configured to perform the method for reconstructing a dose distribution for a region of interest according to any one of the embodiments described above.

The details of the various embodiments of the present disclosure will be illustrated with the accompanying drawings and description below, based on which, other features, problems to be solved, and beneficial effects of the disclosure will be readily understood by those skilled in the art.

### BRIEF DESCRIPTION OF THE DRAWINGS

FIG. 1 is a view of an application environment of a method for reconstructing a dose distribution according to an embodiment;

FIG. 2 is a view of an application environment of a method for reconstructing a dose distribution according to an embodiment;

FIG. 3 is a schematic flow diagram of a method for reconstructing a dose distribution according to an embodiment;

FIG. 4 is a schematic flow diagram of a method for reconstructing a dose distribution according to an embodiment;

FIG. 5 is a schematic flow diagram of a method for reconstructing a dose distribution according to an embodiment;

FIG. 6 is a schematic flow diagram of a method for reconstructing a dose distribution according to an embodiment;

FIG. 7 is a schematic flow diagram of a method for reconstructing a dose distribution according to an embodiment;

FIG. 8 is a schematic flow diagram of a method for reconstructing a dose distribution according to an embodiment;

FIG. 9 is a schematic flow diagram of a method for reconstructing a dose distribution according to an embodiment;

FIG. 10 is a schematic flow diagram of a method for reconstructing a dose distribution according to an embodiment; and

FIG. 11 is a structural block diagram of an apparatus for reconstructing a dose distribution according to an embodiment.

### DETAILED DESCRIPTION

The present disclosure will be described in further detail below with reference to the accompanying drawings and embodiments in order to make the objects, technical solutions, and advantages of the present disclosure more clear. It should be understood that the specific embodiments described herein are only for explaining the present disclosure, and not intended to limit the present disclosure.

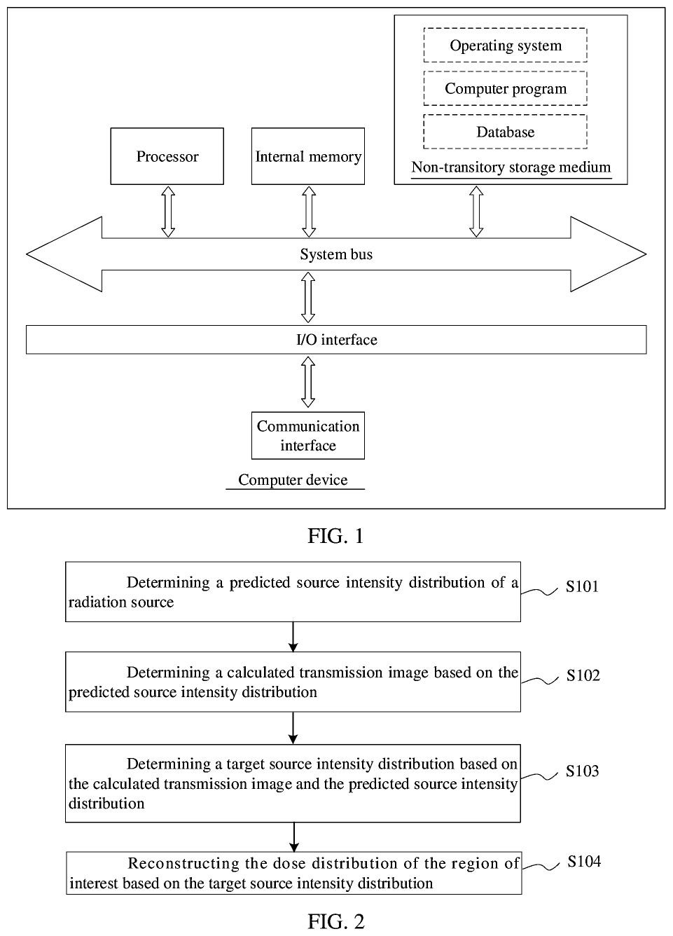

A method for reconstructing a dose distribution according to an embodiment of the present disclosure can be applied to an application environment shown in FIG. 1. For example, a computer device may be a server, a personal computer, a notebook computer, a smartphone, a tablet computer, a smart mobile phone, or the like. The computer device may include a processor, a memory, and a network interface connected by a system bus or wirelessly. The processor of the computer device is configured to provide computing and control capabilities. The memory of the computer device may include a non-transitory storage medium and an internal memory. The non-transitory storage medium stores an operating system, a computer program, and a database. The internal memory provides an environment for running the operating system and the computer program in the non-transitory storage medium. The database of the computer device is configured to store data during dose distribution reconstruction. The network interface of the computer device is configured to communicate with an external terminal through a network connection, and the computer program implements a method for reconstructing a dose distribution when executed by the processor. The computer device can be implemented by an independent controller or a controller cluster composed of a plurality of controllers. It should be noted that the memory of the computer device is not limited to the above memory, and may also include a high-speed random access memory, a volatile solid-state memory, or the like. In addition, the composition architecture of the computer device is not limited to the above architecture, and some components may be added or omitted.

It should be noted that the method for reconstructing a dose distribution according to the present disclosure can be used in a dose monitoring process. In addition to a patient dose quality assurance (QA) process, the method can be used in other scenarios, for example, a phantom quality evaluation process, and if an electronic portal imaging device (EPID), a dose detector or other measuring devices can obtain a transmission dose transmitted through a phantom, a deposited dose in the phantom can also be determined using the method for reconstructing a dose distribution according to the present disclosure. With the method for reconstructing a dose distribution according to the present disclosure, a three-dimensional dose distribution in a target object can be compared and verified more intuitively to provide reference for a subsequent radiotherapy process, thus realizing a dose-guided radiotherapy process.

In an embodiment, as shown in FIG. 2, a method for reconstructing a dose distribution is provided. Taking the example where the method is applied to a computer device shown in FIG. 1, the method includes the following steps:
S101: determining a predicted source intensity distribution of a radiation source;
S102: determining a calculated transmission image based on the predicted source intensity distribution;
S103: determining a target source intensity distribution based on the calculated transmission image and the predicted source intensity distribution; and
S104: reconstructing the dose distribution of the region of interest based on the target source intensity distribution.

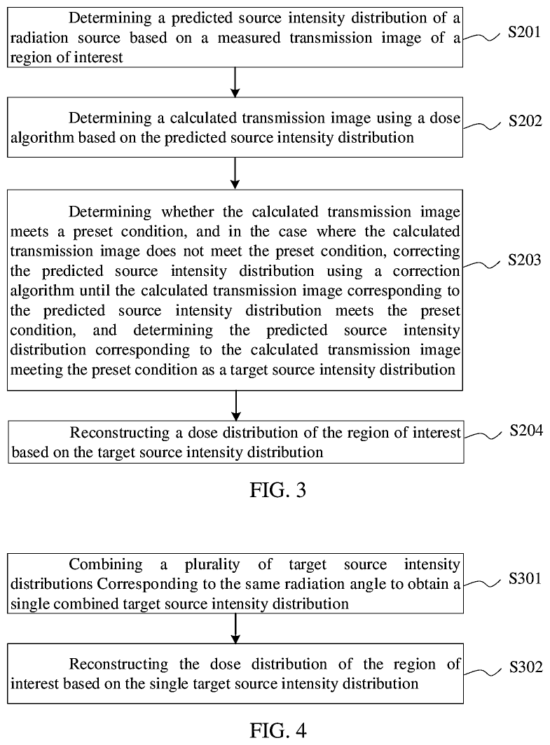

In an embodiment, as shown in FIG. 3, the method includes the following steps:
S201: determining a predicted source intensity distribution of a radiation source based on a measured transmission image of a region of interest.

In general, the radiation source, the region of interest, and a measuring device may be arranged in sequence, that is, the region of interest is arranged between the radiation source and the measuring device. The region of interest may be a human body, a phantom, or a specified part of the human body or phantom. For example, the region of interest includes a target region, a tumor region, or an adjacent tissue, etc. The radiation source refers to a source device that emits a radiation dose and is configured to emit a dose to the region of interest. After the radiation dose passes through the region of interest, one part of the radiation dose is deposited on the region of interest, and the other part of the radiation dose is transmitted or scattered to other regions (for example, may be attached to the measuring device). For example, the measured transmission dose may be a dose transmitted to the measuring device or a dose calculated from the dose transmitted to the measuring device. The measured transmission image of the region of interest may be measured by the measuring device which in turn sends the transmission image to the computer device after measuring the transmission image. The gray values of each pixel in the transmission image reflect the dose distribution after passing through the region of interest. Based on the measured transmission image, a transmission dose distribution can be further obtained. The measured transmission dose distribution contains information, such as a dose value or a dose distribution shape at each detection position. For example, the measuring device may be an EPID, a dose detector, or the like. Alternatively, the computer device may further obtain the measured transmission image and/or the measured transmission dose distribution of the region of interest from a dose distribution server based on identification information of the region of interest.

A source intensity distribution refers to a distribution of the dose in the radiation source (for example, a dose value, a distribution shape, or the like, at each point in a space), a distribution of a dose that can be formed in the space when radiation is emitted from the radiation source, or the like. There exists a mutual mapping relationship between the source intensity distribution and the measured transmission image (for example, this relationship may be determined based on experience, theoretical calculations, or big data calculations). The computer device may extrapolate or determine the source intensity distribution based on the mapping relationship and the measured transmission image and determine the source intensity distribution as the predicted source intensity distribution. Alternatively, the computer device may further input the measured transmission image of the region of interest into a trained neural network model, and the measured transmission image is analyzed by the trained neural network model to determine the predicted source intensity distribution of the radiation source.

In other embodiments, there is a mapping relationship between the source intensity distribution and the measured transmission dose distribution, and the computer device may extrapolate or determine the source intensity distribution based on the mapping relationship and the measured transmission dose distribution, and determine it as the predicted source intensity distribution. Alternatively, the measured transmission dose distribution may be input into a trained neural network model, and the measured transmission dose distribution may be analyzed by the trained neural network model to determine the predicted source intensity distribution of the radiation source.

It should be noted that the radiation source may irradiate the region of interest with the dose from a single radiation angle corresponding to one measured transmission image. The radiation may also be performed from different radiation angles, and the measured transmission images obtained from different radiation angles may be different. That is, one radiation angle corresponds to one measured transmission image, and therefore, the measured transmission image includes a plurality of transmission images measured at different radiation angles of the radiation source. Correspondingly, the predicted source intensity distribution includes a plurality of predicted source intensity distributions of the radiation source at the radiation angles. The source intensity distribution can be determined more accurately by acquiring the plurality of transmission images measured at different radiation angles and determining the predicted source intensity distribution based thereon.
S202: determining a calculated transmission image using a dose algorithm based on the predicted source intensity distribution.

The calculated image refers to a theoretical transmission image on the measuring device after the dose is transmitted through the region of interest when the source intensity distribution of the radiation source is the predicted source intensity distribution. That is, the calculated transmission image is a theoretical image. A calculated transmission dose distribution can be then obtained based on the calculated transmission image. The dose algorithm includes a mapping relationship between the source intensity distribution and the calculated transmission image, and/or a mapping relationship between the source intensity distribution and the calculated transmission dose distribution. For example, the dose algorithm may be a Monte Carlo (MC) dose calculation engine, or the like. Furthermore, the dose algorithm may also be a convolution algorithm, a pencil beam algorithm, or other types of dose algorithms, and the type of the dose algorithm is not limited in the present embodiment.

It is to be understood that the mapping relationship between the source intensity distribution and the calculated transmission image and/or calculated transmission dose distribution is not the same as the mapping relationship between the source intensity distribution and the measured transmission image and/or measured transmission dose distribution described in step S201. For example, the mapping relationship between the source intensity distribution and the measured transmission image may be obtained based on actual data, while the mapping relationship between the source intensity distribution and the calculated transmission image represents a theoretical relationship between the two.

In the present embodiment, since the dose algorithm includes the mapping relationship between the source intensity distribution and the transmission image, the computer device may analyze the predicted source intensity distribution using the dose algorithm, and determine the calculated transmission image corresponding to each predicted source intensity distribution, so as to obtain the calculated transmission image corresponding to the predicted source intensity distribution.

In other embodiments, the predicted source intensity distribution of the radiation source may be determined in other ways. For example, a preset source intensity distribution is obtained as an initial predicted source intensity distribution. The preset source intensity distribution may be obtained based on a hardware configuration of the radiation source.
S203: determining whether the calculated transmission image meets a preset condition, and in the case that the calculated transmission image does not meet the preset condition, correcting the predicted source intensity distribution using a correction algorithm until the calculated transmission image corresponding to the corrected predicted source intensity distribution meets the preset condition, and determining the predicted source intensity distribution corresponding to the calculated transmission image meeting the preset condition as a target source intensity distribution.

It is to be understood that the correction of the predicted source intensity distribution may include one or more corrections, and the calculated transmission image meeting the preset condition includes a calculated transmission image meeting the preset condition calculated based on the predicted source intensity distribution or a calculated transmission image meeting the preset condition calculated based on the corrected predicted source intensity distribution.

The correction algorithm may include a correction algorithm based on a reference dose distribution model (for example, a Monte Carlo dose model, a pencil beam dose model, or the like), a machine learning model (for example, a supervised learning model, a semi-supervised learning model, a reinforcement learning model, or the like), or other types of correction algorithms or models. For example, the reference dose distribution model for serving as a reference object may be configured to preliminarily correct the predicted source intensity distribution. For example, the machine learning model may be configured to post-correct the predicted source intensity distribution. As an example, when the correction algorithm includes the reference dose distribution model and the machine learning model, the predicted source intensity distribution is preliminarily corrected by the reference dose distribution model, and then, the preliminarily corrected predicted source intensity distribution is further corrected by the machine learning model based on the preliminary correction. As an example, the machine learning model may take, as a learning target, a source intensity distribution generated when a radiotherapy planning system performs a forward calculation. Further, the smoothness of the source intensity distribution can be improved by performing further correction through the machine learning model, thus effectively enhancing a tongue&groove (T&G) effect of a multi-leaf collimator and a leaftip effect of the multi-leaf collimator. In addition, when the smoothness of the source intensity distribution is improved, the source intensity distribution may be corrected using a smoothing correction algorithm. For example, the smoothing correction algorithm may be a virtual space smoothing algorithm, an absolute discounting method, or the like. Furthermore, without limitation, the preliminary correction and/or the further correction may be performed by a user or using other correction algorithms.

In the present embodiment, the computer device may analyze the calculated transmission dose distribution to determine whether the calculated transmission image meets the condition. When the calculated transmission image is determined to meet the preset condition, the dose distribution of the region of interest is directly reconstructed based on the predicted source intensity distribution without correcting the predicted source intensity distribution.

When the calculated transmission image is determined not to meet the preset condition, the predicted source intensity distribution is continuously corrected using the correction algorithm (for example, the predicted source intensity distribution may be input into a correction model for correction). After each correction is completed, the calculated transmission dose corresponding to the corrected predicted source intensity distribution is obtained, and whether the calculated transmission dose meets the preset condition is continuously judged. If yes, the correction is stopped, and the corrected predicted source intensity distribution is determined as the target source intensity distribution; and if no, a next correction is continuously performed using the correction algorithm.

As a non-limiting example, the preset condition may include whether a difference between the calculated transmission image and the measured transmission image (for example, a difference matrix or a difference at a particular point) is less than a preset threshold. As another non-limiting example, the preset condition may be whether a difference matrix between a calculated dose image (for example, an EPID image) and a measured dose image corresponding to the measured transmission image passes a convergence condition. Herein, the preset condition is not particularly limited, and may be determined according to actual situations and requirements.
S204: reconstructing a dose distribution of the region of interest based on the target source intensity distribution.

For example, the dose distribution of the region of interest refers to the distribution of the dose deposited or otherwise kept at the region of interest, which contains information, such as dose values or dose distribution shapes at various locations.

In the present embodiment, the computer device may reconstruct the dose distribution (for example, a three-dimensional dose distribution, a two-dimensional dose distribution, or the like) of the region of interest corresponding to each of the target source intensity distributions based on the preset relationship between the source intensity distribution and the dose distribution at the region of interest.

It should be noted that, when the number of the target source intensity distributions is plural, the dose distributions of the region of interest corresponding to all the target source intensity distributions may be sequentially determined, and the plurality of dose distributions of the region of interest are combined to obtain the final dose distribution of the region of interest. Alternatively, the computer device may also combine the plurality of target source intensity distributions corresponding to the same radiation angle into a single combined target source intensity distribution corresponding to this radiation angle, and determine the dose distribution of the region of interest based on the combined target source intensity distribution. For example, the plurality of target source intensity distributions may be combined into the single target source intensity distribution in various ways, such as weighted addition and direct superposition.

In the method for reconstructing a dose distribution, the predicted source intensity distribution of the radiation source is determined; the calculated transmission image is determined using the dose algorithm based on the predicted source intensity distribution; whether the calculated transmission image meets the preset condition is determined, and in the case that the calculated transmission image does not meet the preset condition, the predicted source intensity distribution is corrected using the correction algorithm until the calculated transmission image corresponding to the predicted source intensity distribution meets the preset condition, and the predicted source intensity distribution corresponding to the calculated transmission image meeting the preset condition is taken as the target source intensity distribution; and the dose distribution of the region of interest is reconstructed based on the target source intensity distribution. In the method, the calculated transmission image can be determined based on the predicted source intensity distribution. The condition judgment is performed on the calculated transmission image, and the initial predicted source intensity distribution is continuously corrected based on the judgment result. The corrected target source intensity distribution can truly reflect the source intensity distribution of the radiation source. The obtained target source intensity distribution is more accurate, and the dose distribution of the region of interest can be more accurately obtained based on the target source intensity.

On the basis of the above embodiments, the following embodiment describes details of the step S204 of reconstructing the dose distribution of the region of interest based on the target source intensity distribution in FIG. 3. As shown in FIG. 4, as a non-limiting example, the step S204 may include:
S301: in a case that the target source intensity distribution includes a plurality of target source intensity distributions corresponding to the same radiation angle of the radiation source, combining the plurality of target source intensity distributions to obtain a single combined target source intensity distribution.

In the present embodiment, the plurality of target source intensity distributions are target source intensity distributions of the radiation source corresponding to the same radiation angle respectively. The computer device may add the plurality of target source intensity distributions, and take the target source intensity distribution obtained by the addition as the single combined target source intensity distribution.

The target source intensity distributions may be obtained for different radiation angles, and for each of the different radiation angles, a plurality of target source intensity distributions can be obtained. The combination of the plurality of source intensity distributions may be performed for each radiation angle, such that a plurality of combined target source intensity distributions can be obtained, respectively.
S302: reconstructing the dose distribution of the region of interest based on the combined target source intensity distribution.

In the above method for reconstructing a dose distribution, the plurality of target source intensity distributions are combined to obtain the single combined target source intensity distribution; the dose distribution of the region of interest is reconstructed based on the single target source intensity distribution. In the method, the combined target source intensity distribution can be obtained by combining the plurality of target source intensity distributions, and therefore, the dose distribution of the region of interest can be reconstructed more accurately.

In other embodiments, the step S204 may include:
obtaining a plurality of dose distribution of the regions of interest corresponding to the plurality of target source intensity distributions, respectively; and
combining the plurality of dose distributions of the region of interest to obtain a combined dose distribution of the regions of interest.

The plurality of target source intensity distributions here refer to target source intensity distributions of the radiation source corresponding to different radiation angles. The region of interest will have a deposited dose under any of the different radiation angles. In the above method, the plurality of dose distributions of the region of interest are combined to obtain the combined dose distribution of the region of interest, thereby making the dose distribution of the region of interest more accurate.

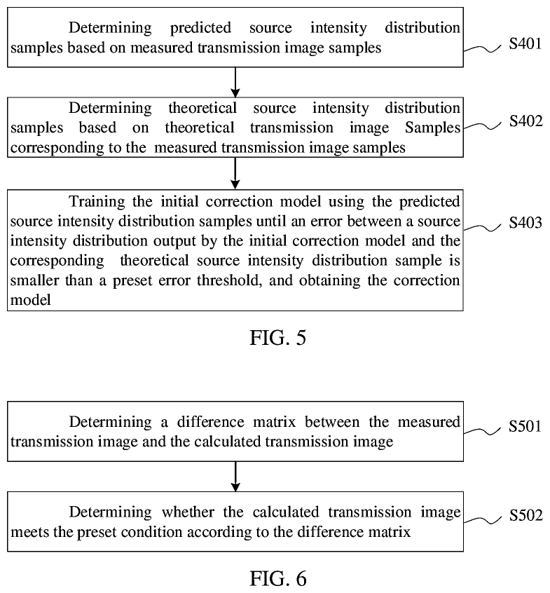

On the basis of the above embodiments, the following embodiment describes details of a training process of the correction model used in the correction algorithm. As shown in FIG. 5, as a non-limiting example, the training process may include:
S401: determining predicted source intensity distribution samples based on measured transmission image samples.

In the present embodiment, before an initial correction model is trained, training samples need to be determined. The computer device may acquire a plurality of historical measured transmission image samples and determine sample predicted source intensity distribution samples based on the historical measured transmission image samples.

For example, a measured transmission dose distribution corresponding to each of the historical measured transmission images is determined. The computer device determines a predicted source intensity distribution corresponding to each historical measured transmission image based on the measured transmission dose distribution and the mapping relationship between the measured transmission dose distribution and a source intensity distribution, and determines the obtained plurality of predicted source intensity distributions as predicted source intensity distribution samples.
S402: determining theoretical source intensity distribution samples based on theoretical transmission image samples corresponding to the measured transmission image samples.

In the present embodiment, the computer device may determine the corresponding theoretical transmission image samples according to the measured transmission image samples, and then analyze the theoretical transmission image samples to determine theoretical transmission dose distribution samples.

For example, the measured transmission dose distribution samples are obtained based on the measured transmission image samples, and the theoretical transmission dose distribution samples and the corresponding theoretical transmission image samples are obtained based on the previously described mapping relationship between the source intensity distribution and the measured transmission dose distribution, and the mapping relationship between the source intensity distribution and the calculated transmission dose distribution. Then, the computer device determines the theoretical source intensity distribution samples corresponding to the theoretical transmission image samples according to the theoretical transmission dose distribution samples.
S403: training the initial correction model using the predicted source intensity distribution samples.

In some embodiments, the initial correction model is trained using the predicted source intensity distribution samples until an error between a source intensity distribution output by the initial correction model and the corresponding theoretical source intensity distribution sample is smaller than a preset error threshold, and obtaining the correction model.

In the present embodiment, the computer device may train the initial correction model using the predicted source intensity distribution samples, and calculate the error between the output source intensity distribution and the corresponding theoretical source intensity distribution sample after the source intensity distribution is output by the initial correction model. The error is compared with the preset error threshold, and the initial correction model is continuously trained when the error is determined to be greater than the preset error threshold. When the error is determined to be smaller than the preset error threshold, the correction of the initial correction model is stopped, and the correction model is obtained.

In the method for reconstructing a dose distribution, the predicted source intensity distribution samples are determined based on the measured transmission image samples; the theoretical source intensity distribution samples are determined based on the theoretical transmission image samples corresponding to the measured transmission image samples; the initial correction model is trained using the predicted source intensity distribution samples until the error between the source intensity distribution output by the initial correction model and the corresponding theoretical source intensity distribution sample is smaller than the preset error threshold, and the correction model is obtained. In the method, the initial correction model is trained using the predicted source intensity distribution samples corresponding to the measured transmission image samples, and a training termination condition is determined based on the theoretical source intensity distribution samples, so that the obtained correction model can correct a predicted source intensity distribution more accurately.

On the basis of the above embodiment, the following embodiment describes details of the step S203 of determining whether the calculated transmission image meets the preset condition in FIG. 3. As shown in FIG. 6, as a non-limiting example, the step S203 may include:
S501: determining a difference matrix between the measured transmission image and the calculated transmission image.

In the present embodiment, one measured transmission image corresponds to one calculated transmission image. For any measured transmission image and the corresponding calculated transmission image, the computer device may calculate a difference between the measured transmission image and the calculated transmission image. The difference may be in the form of a difference matrix, elements of which include, for example, gray differences between corresponding pixels of the measured transmission image and the calculated transmission image.

In other embodiments, a difference matrix between the measured transmission dose distribution and the calculated transmission dose distribution is determined. Specifically, the computer device may calculate a difference matrix between the measured transmission dose distribution and the calculated transmission dose distribution. Elements in the difference matrix include, for example, dose differences at corresponding locations in the dose distribution.
S502: determining whether the calculated transmission image meets the preset condition according to the difference matrix.

In the present embodiment, a large difference matrix indicates that the calculated transmission image is inaccurate, and the calculated transmission image is required to be corrected. A small difference matrix indicates that the calculated transmission image is accurate, and the calculated transmission image is not required to be corrected. The computer device may compare the difference matrix with a preset difference matrix and determine whether the transmission image meets the preset condition according to the comparison result. Alternatively, the computer device may further judge whether the difference matrix is within a matrix range needing correction, and determine whether the calculated transmission dose meets the preset condition according to the judgment result. For example, if the difference matrix is within the matrix range needing correction, the calculated transmission image is determined not to meet the preset condition, and if the difference matrix is not within the matrix range needing correction, the calculated transmission image is determined to meet the preset condition. It is to be understood that a value of the difference matrix can be determined according to one or more elements in the difference matrix based on a predefined rule.

In the above method for reconstructing a dose distribution, the difference matrix between the measured transmission image and the calculated transmission image is determined, and whether the calculated transmission image meets the preset condition is determined according to the difference matrix. In the method, whether the calculated transmission image is accurate can be accurately determined based on the difference matrix of the measured transmission image and the calculated transmission image, and therefore, whether the calculated transmission image meets the preset condition can be determined accurately.

Similarly, in other embodiments, a difference matrix between the measured transmission dose distribution and the calculated transmission dose distribution may be determined, and based on the difference matrix, whether the calculated transmission dose distribution meets the preset condition is determined.

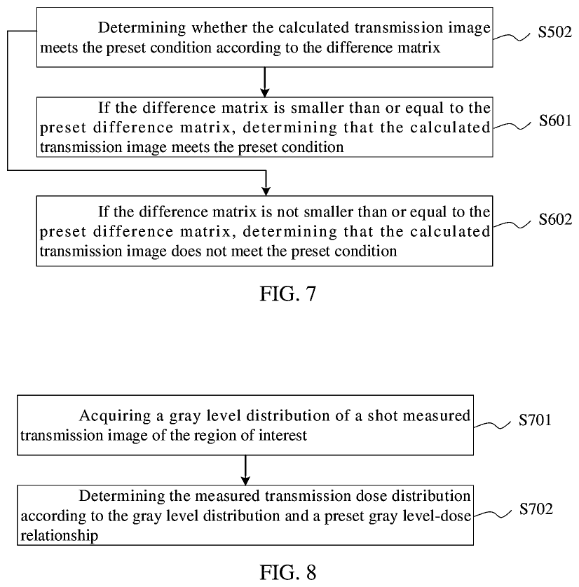

On the basis of the above embodiment, the following embodiment describes details of the step S502 of determining whether the calculated transmission image meets the preset condition according to the difference matrix” in FIG. 6. As shown in FIG. 7, as a non-limiting example, the step S502 may include:
S601: if the difference matrix is smaller than or equal to the preset difference matrix, determining that the calculated transmission image meets the preset condition.

In the present embodiment, the computer device may compare the difference matrix with the preset difference matrix, and if the difference matrix is smaller than or equal to the preset difference matrix, the difference between the measured transmission image and the calculated transmission image is small. In this case, the calculated transmission image is determined to meet the preset condition without any correction requirement.
S602: otherwise, determining that the calculated transmission image does not meet the preset condition.

In the present embodiment, if the difference matrix is not smaller than or equal to the preset difference matrix, the difference between the measured transmission image and the calculated transmission image is large. In this case, the calculated transmission image is determined not to meet the preset condition and needs correction.

In the above method for reconstructing a dose distribution, if the difference matrix is smaller than or equal to the preset difference matrix, the calculated transmission image is determined to meet the preset condition, and if the difference matrix is larger than the preset difference matrix, the calculated transmission dose distribution is determined not to meet the preset condition. In the method, the difference matrix is compared with the preset difference matrix, and whether the calculated transmission image meets the condition or not can be accurately determined according to different comparison results.

In some embodiments, the comparison between the difference matrix and the preset difference matrix in the above method includes comparing any element in the difference matrix with a corresponding element in the preset difference matrix. When all the elements of the difference matrix are smaller than or equal to the corresponding elements of the preset difference matrix, the difference matrix is smaller than or equal to the preset difference matrix. Otherwise, the difference matrix is not smaller than or equal to the preset difference matrix.

It will be appreciated that the above method of determining whether the preset condition is met based on the difference matrix is equally applicable to the judgment of the calculated transmission dose distribution. If the difference matrix is less than or equal to the preset difference matrix, it is determined that the calculated transmission dose distribution meets the preset condition. Otherwise, it is determined that the calculated transmission dose distribution does not meet the preset condition.

On the basis of the above embodiment, the following embodiment describes details of the determination of the measured transmission image. As shown in FIG. 8, as a non-limiting example, the method may further include:
S701: acquiring a gray distribution of a measured transmission image of the region of interest.

The gray distribution refers to a distribution of gray values of pixels of the measured transmission image.

In the present embodiment, after the computer device acquires the measured transmission image corresponding to the region of interest, the gray value of each pixel of the measured transmission image may be extracted using a gray feature extraction algorithm, and the gray distribution of the measured transmission image is determined according to the gray value of each pixel.
S702: determining the measured transmission dose distribution according to the gray distribution and a preset gray-dose relationship.

The preset gray-dose relationship refers to a relationship between the gray value of each pixel and the corresponding dose. For one pixel, the larger the gray value, the higher the corresponding dose, and the smaller the gray value, the smaller the corresponding dose.

In the present embodiment, the computer device may determine the dose corresponding to each gray value according to the preset gray-dose relationship, so as to obtain a dose distribution corresponding to the gray distribution of the measured transmission image, and determine the dose distribution as the measured transmission dose distribution.

In the above method for reconstructing a dose distribution, the gray distribution of the measured transmission image of the region of interest is acquired, and the measured transmission dose distribution is determined according to the gray distribution and the preset gray-dose relationship. In the method, the gray value of the measured transmission image is analyzed, and the measured transmission dose distribution corresponding to the gray value of the measured transmission image can be accurately determined according to the preset gray-dose relationship.

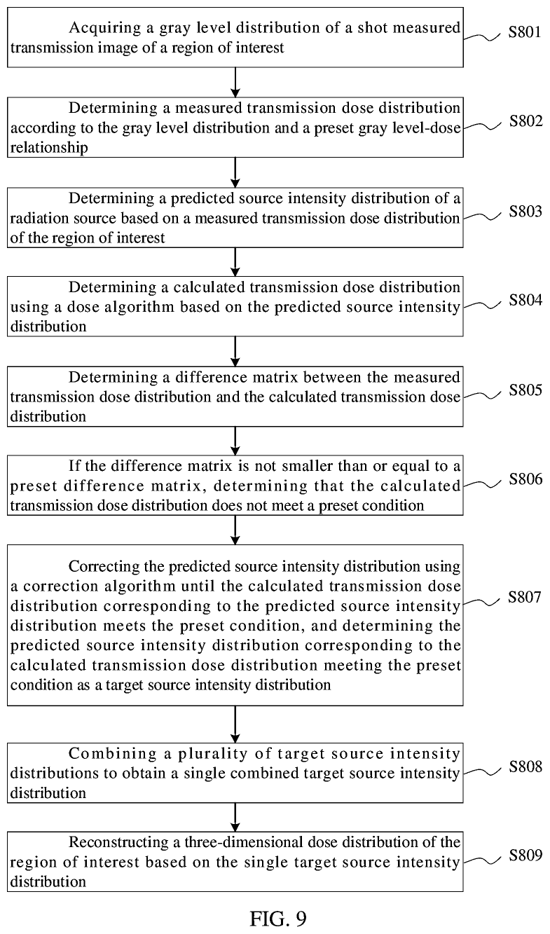

A method for reconstructing a dose distribution according to an embodiment is described as follows. As shown in FIG. 9, the method may include:
S801: acquiring a gray distribution of a measured transmission image of a region of interest;
S802: determining a measured transmission dose distribution according to the gray distribution and a preset gray-dose relationship;
S803: determining a predicted source intensity distribution of a radiation source based on a measured transmission dose distribution of the region of interest;
S804: determining a calculated transmission dose distribution using a dose algorithm based on the predicted source intensity distribution;
S805: determining a difference matrix between the measured transmission dose distribution and the calculated transmission dose distribution;
S806: if the difference matrix is not smaller than or equal to a preset difference matrix, determining that the calculated transmission dose distribution does not meet a preset condition;
S807: correcting the predicted source intensity distribution using a correction algorithm until the calculated transmission dose distribution corresponding to the corrected predicted source intensity distribution meets the preset condition, and determining the predicted source intensity distribution corresponding to the calculated transmission dose distribution meeting the preset condition as a target source intensity distribution;
S808: combining a plurality of target source intensity distributions to obtain a single combined target source intensity distribution; and
S809: reconstructing a dose distribution of the region of interest based on the single target source intensity distribution.

Therefore, in the method according to some embodiments of the present disclosure, the calculated transmission image is determined based on the predicted source intensity distribution. The target source intensity distribution is then determined based on the calculated transmission image, and the dose distribution of the region of interest is reconstructed based on the target source intensity distribution. In further embodiments, the predicted source intensity can be determined based on the measured transmission images, and/or the accuracy of the predicted source intensity can be assessed by comparing the measured transmission image with the calculated transmission image.

In other embodiments of the dose distribution reconstruction method, the measured transmission dose distribution can be obtained based on the measured transmission image, and/or the calculated transmission dose distribution can be obtained based on the calculated transmission image. As previously described, these dose distributions can be applied in the dose distribution reconstruction method. Therefore, the preset disclosure also provides a method for reconstructing a dose distribution, including:
determining a predicted source intensity distribution of a radiation source;
determining a calculated transmission dose distribution based on the predicted source intensity distribution;
determining a target source intensity distribution based on the calculated transmission dose distribution and the predicted source intensity distribution; and
reconstructing the dose distribution of the region of interest based on the target source intensity distribution.

In some embodiments, the predicted source intensity distribution for the radiation source is determined based on a measured transmission dose distribution of the region of interest.

In some embodiments, the predicted source intensity distribution is corrected using a correction algorithm to obtain the target source intensity distribution.

In some embodiments, the method further includes training the correction model, including training an initial correction model using predicted source intensity distribution samples until an error between a source intensity distribution output from the initial correction model and the corresponding theoretical source intensity distribution is less than a preset error threshold, to obtain the correction model.

In some embodiments, determining the target source intensity distribution based on the transmission dose distribution and the predicted source intensity distribution includes determining whether the calculated transmission dose distribution meets a preset condition, in the case that the calculated transmission dose distribution does not meet the preset condition, correcting the predicted source intensity distribution using the correction algorithm until the calculated transmission dose distribution corresponding to the predicted source intensity distribution meets the preset condition, and determining the predicted source corresponding to the calculated transmission dose distribution meeting the preset condition as the target source intensity distribution.

In some embodiments, determining whether the calculated transmission dose distribution meets the preset condition includes determining a difference matrix between the measured transmission dose distribution and the calculated transmission dose distribution, and determining whether the calculated transmission dose distribution meets the preset condition based on the difference matrix.

In some embodiments, before determining the predicted source intensity distribution of the radiation source based on the measured transmission dose distribution of the region of interest, the method includes obtaining a gray distribution of the measured transmission image of the region of interest, and determining the measured transmission dose distribution based on the gray distribution and a preset gray-dose relationship.

In some embodiments, the calculated transmission dose distribution is determined based on the predicted source intensity distribution through a dose algorithm. The dose algorithm including a Monte Carlo dose algorithm, and/or the correction algorithm includes a machine learning algorithm.

In some embodiments, a plurality of target source intensity distributions at the same radiation angle are obtained, and the plurality of target source intensity distributions are combined to obtain a single combined target source intensity distribution. The dose distribution of the region of interest is reconstructed based on the single combined target source intensity distribution.

In some embodiments, a plurality of target source intensity distributions corresponding to different radiation angles are obtained, and a plurality of dose distributions of the region of interest are obtained based on the plurality of target source intensity distributions. The plurality of dose distributions are combined to obtain a combined dose distribution of the region of interest.

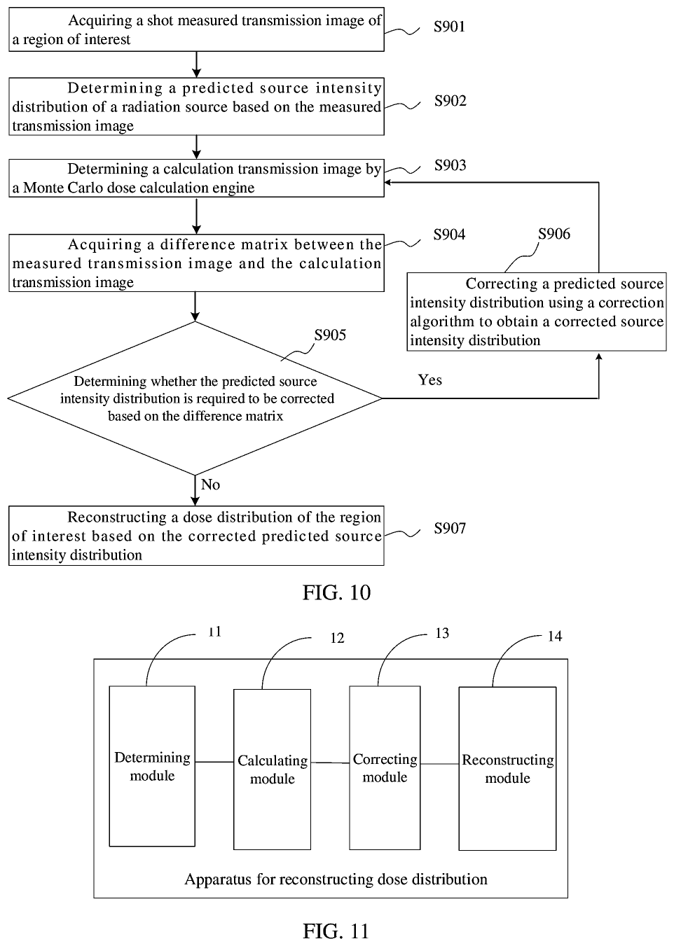

FIG. 10 is a schematic flow diagram of the method for reconstructing a dose distribution according to an embodiment, and the method includes:
S901: acquiring a measured transmission image of a region of interest;
S902: determining a predicted source intensity distribution of a radiation source based on the measured transmission image;
S903: determining a calculation transmission image by a Monte Carlo dose calculation engine;
S904: acquiring a difference matrix between the measured transmission image and the calculation transmission image;
S905: determining whether the predicted source intensity distribution is required to be corrected based on the difference matrix, if correction is required, executing step S906, and if no correction is required, executing step S907;
S906: correcting the predicted source intensity distribution using a correction algorithm to obtain a corrected source intensity distribution, and continuing to execute step S903; and
S907: reconstructing a dose distribution of the region of interest based on the corrected predicted source intensity distribution.

It can be understood that the dose reconstruction method provided in various embodiments of the present disclosure can be applied to a human body for monitoring the radiation dose during a radiation process, as well as to a phantom. In the present disclosure, the transmission image generated by the transmitted dose passing through the phantom and obtained from dose detectors or other measurement devices can be used to determine the deposited dose within the phantom, thus providing reference for subsequent radiation therapy processes and enabling dose-guided radiation therapy. Therefore, in some embodiments, the region of interest includes the phantom or a specified part of the phantom. Furthermore, the reconstructed dose distribution can be compared with a planned dose distribution, and an adjustment to a radiation scheme can be made based on the comparison result to achieve dose control for the patient. The planned dose distribution includes a planned dose distribution generated by the radiotherapy planning system or a preset dose distribution.

It should be understood that, although the steps in the flow charts involved in the above embodiments are shown in sequence as indicated by the arrows, the steps are not necessarily performed in sequence as indicated by the arrows. Unless explicitly stated herein, the steps are not limited to being performed in the exact order and may be performed in other orders. At least part of the steps in the flow charts involved in the above embodiments may include multiple steps or multiple stages, which are not necessarily performed at the same moment, but may be performed at different moments, and the steps or the stages are not necessarily performed in sequence, but may be performed alternately with other steps or at least part of the steps or the stages in other steps.

Based on the same inventive concept, the embodiment of the present disclosure further provides an apparatus for reconstructing a dose distribution, which implements the above methods for reconstructing a dose distribution. The implementation solution for solving problems provided by the apparatus is similar to the implementation solution described in the above methods, and therefore, for the specific definitions in one or more embodiments of the apparatus for reconstructing a dose distribution provided below, reference can be made to the definitions of the methods for reconstructing a dose distribution in the above description, and the definitions are not repeated herein.

In an embodiment, as shown in FIG. 11, there is provided an apparatus for reconstructing a dose distribution, including a determining module 11, a calculating module 12, a correcting module 13, and a reconstructing module 14.

The determining module is configured to determine a predicted source intensity distribution of a radiation source based on a measured transmission image of a region of interest.

The calculating module is configured to determine a calculated transmission image using a dose algorithm based on the predicted source intensity distribution.

The measured transmission image includes a plurality of transmission images measured at different radiation angles of the radiation source, and the predicted source intensity distribution includes a plurality of predicted source intensity distributions of the radiation source at the radiation angles.

The correcting module is configured to determine whether the calculated transmission image meets a preset condition, and in the case that the calculated transmission image does not meet the preset condition, correct the predicted source intensity distribution using a correction algorithm until the calculated transmission image corresponding to the predicted source intensity distribution meets the preset condition, and take the predicted source intensity distribution corresponding to the calculated transmission image meeting the preset condition as a target source intensity distribution.

The dose algorithm includes a Monte Carlo dose algorithm and/or the correction algorithm includes a machine learning model.

The reconstructing module is configured to reconstruct a dose distribution of the region of interest based on the target source intensity distribution.

In an embodiment, the reconstructing module includes a combining unit and a reconstructing unit.

The combining unit is configured to combine the plurality of target source intensity distributions to obtain a single combined target source intensity distribution.

The reconstructing unit is configured to reconstruct the dose distribution of the region of interest based on the single target source intensity distribution.

In an embodiment, the above apparatus for reconstructing a dose distribution further includes a first sample determining module, a second sample determining module, and a training module.

The first sample determining module is configured to determine a predicted source intensity distribution sample based on a measured transmission image sample.

The second sample determining module is configured to determine a theoretical source intensity distribution sample based on a theoretical transmission image sample corresponding to the measured transmission image sample.

The training module is configured to train the initial correction model using the predicted source intensity distribution samples until an error between a source intensity distribution output by the initial correction model and the theoretical source intensity distribution sample is smaller than a preset error threshold, and obtain the correction model.

In an embodiment, the correcting module further includes a first determining unit and a second determining unit.

The first determining unit is configured to determine a difference matrix between the measured transmission image and the calculated transmission image.

The second determining unit is configured to determine whether the calculated transmission image meets the preset condition according to the difference matrix.

In an embodiment, the second determining unit is further configured to, in the case that the difference matrix is smaller than or equal to the preset difference matrix, determine that the calculated transmission image meets the preset condition; and otherwise, determine that the calculated transmission image does not meet the preset condition.

In an embodiment, the above apparatus for reconstructing a dose distribution further includes an acquiring module and a image determining module.

The acquiring module is configured to acquire a gray distribution of a measured transmission image of the region of interest.

The image determining module is configured to determine the measured transmission image according to the gray distribution and a preset gray-dose relationship.

The modules in the apparatus for reconstructing a dose distribution may be wholly or partially implemented by software, hardware, and a combination thereof. The modules may be embedded in or independent of the processor in the computer apparatus in hardware, or may be stored in the memory in the computer apparatus in software, such that the processor can conveniently call the modules to execute the operations corresponding to the modules.

In an embodiment, there is provided a computer apparatus, including a memory storing a computer program and a processor. The processor, when executing the computer program, is configured to perform any embodiment of the method for reconstructing a dose distribution described above.

In an embodiment, there is provided a non-transitory computer-readable storage medium having a computer program stored thereon. The computer program, when executed by a processor, causes the processor to perform any embodiment of the method for reconstructing a dose distribution described above.

In an embodiment, there is provided a computer program product including a computer program which, when executed by a processor, causes a processor to perform any embodiment of the method for reconstructing a dose distribution described above.

It should be noted that user information (including but not limited to user apparatus information, user personal information, or the like) and data (including but not limited to data for analysis, stored data, displayed data, or the like) involved in the present disclosure are information and data authorized by the user or sufficiently authorized by each party.

It will be understood by those skilled in the art that all or part of the processes of the methods according to the embodiments described above may be implemented by a computer program instructing related hardware, and the computer program may be stored in a non-transitory computer-readable storage medium, and when executed, may include the processes of the embodiments of the methods described above. Any reference to memories, databases or other media used in the embodiments of the present disclosure can include at least one of a non-transitory memory and a volatile memory. The non-transitory memory may include a read-only memory (ROM), a magnetic tape, a floppy disk, a flash memory, an optical memory, a high-density embedded non-transitory memory, a resistive random access memory (ReRAM), a magnetoresistive random access memory (MRAM), a ferroelectric random access memory (FRAM), a phase change memory (PCM), a graphene memory, or the like. The volatile memory can include a random access memory (RAM), an external cache memory, or the like. By way of illustration and not limitation, the RAM can take many forms, such as a static random access memory (SRAM), a dynamic random access memory (DRAM), or the like. The databases involved in the embodiments of the present disclosure may include at least one of relational and non-relational databases. The non-relational database may include, but is not limited to, a block chain-based distributed database, or the like. The processors referred to in the embodiments of the present disclosure may include, but are not limited to, general processors, central processors, graphics processors, digital signal processors, programmable logic units, data processing logic units based on quantum calculations, or the like.

The technical features of the above-mentioned embodiments can be combined arbitrarily. In order to make the description concise, not all possible combinations of the technical features are described in the embodiments. However, as long as there is no contradiction in the combination of these technical features, the combinations should be considered within the scope of the specification.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure. Therefore, the protection scope of the present disclosure should be subject to the appended claims.

What is claimed is:

1. A method for reconstructing a dose distribution for a region of interest, comprising:
determining a predicted source intensity distribution of a radiation source;
determining a calculated transmission image based on the predicted source intensity distribution;
determining a target source intensity distribution based on the calculated transmission image and the predicted source intensity distribution; and
reconstructing the dose distribution of the region of interest based on the target source intensity distribution.

2. The method according to claim 1, wherein determining the predicted source intensity distribution of the radiation source comprises:
determining the predicted source intensity distribution of the radiation source based on a measured transmission image.

3. The method according to claim 1, wherein determining the target source intensity distribution based on the calculated transmission image and the predicted source intensity distribution comprises:
correcting the predicted source intensity distribution to obtain the target source intensity distribution.

4. The method according to claim 3, wherein correcting the predicted source intensity distribution to obtain the target source intensity distribution comprises:
determining whether the calculated transmission image meets a preset condition, and in the case that the calculated transmission image does not meet the preset condition, correcting the predicted source intensity distribution, such that the calculated transmission image corresponding to the corrected predicted source intensity distribution meets the preset condition, and determining the corrected predicted source intensity distribution corresponding to the calculated transmission image meeting the preset condition as the target source intensity distribution.

5. The method according to claim 4, wherein the correction is performed by a machine learning-based correction model.

6. The method according to claim 4, wherein determining the predicted source intensity distribution of the radiation source comprises determining the predicted source intensity distribution of the radiation source based on a measured transmission image, and
wherein determining whether the calculated transmission image meets the preset condition comprises:
determining a difference between the measured transmission image and the calculated transmission image; and
determining whether the calculated transmission image meets the preset condition according to the difference.

7. The method according to claim 6, wherein the difference comprises a difference matrix, and elements of the difference matrix comprise gray differences between corresponding pixels of the measured transmission image and the calculated transmission image, and
wherein determining whether the calculated transmission image meets the preset condition according to the difference comprises:
if the difference matrix is smaller than or equal to a preset difference matrix, determining that the calculated transmission image meets the preset condition; or
otherwise, determining that the calculated transmission image does not meet the preset condition.

8. The method according to claim 5, further comprising training the correction model, wherein training the correction model comprises:
training an initial correction model using predicted source intensity distribution samples to obtain the correction model.

9. The method according to claim 1, wherein determining the calculated transmission image based on the predicted source intensity distribution comprises determining the calculated transmission image using a dose algorithm based on the predicted source intensity distribution, and the dose algorithm comprises a Monte Carlo dose algorithm.

10. The method according to claim 1, wherein the target source intensity distribution comprises a plurality of target source intensity distributions corresponding to different radiation angles of the radiation source respectively, and
wherein reconstructing the dose distribution of the region of interest based on the target source intensity distribution comprises:
obtaining a plurality of dose distributions of the region of interest corresponding to the plurality of target source intensity distributions respectively; and
combining the plurality of dose distributions to obtain a combined dose distribution of the region of interest.

11. The method according to claim 1, wherein the target source intensity distribution comprises a plurality of target source intensity distributions corresponding to a same radiation angle of the radiation source respectively, and
wherein reconstructing the dose distribution of the region of interest based on the target source intensity distribution comprises:
combining the plurality of target source intensity distributions to obtain a combined target source intensity distribution; and
obtaining the dose distribution of the region of interest based on the combined target source intensity distribution.

12. The method according to claim 1, wherein the region of interest comprises a phantom or a specified part of the phantom, and the method further comprises:
comparing the reconstructed dose distribution of the region of interest with a planned dose distribution.

13. A non-transitory computer-readable storage medium having a computer program stored thereon, wherein the computer program, when executed by a processor, causes the processor to perform the method for reconstructing a dose distribution for a region of interest according to claim 1.

14. A computer device, comprising a memory storing a computer program and a processor, wherein the processor, when executing the computer program, is configured to perform a method for reconstructing a dose distribution for a region of interest, the method comprising:
determining a predicted source intensity distribution of a radiation source;
determining a calculated transmission image based on the predicted source intensity distribution;
determining a target source intensity distribution based on the calculated transmission image and the predicted source intensity distribution; and
reconstructing the dose distribution of the region of interest based on the target source intensity distribution.

15. The computer device according to claim 14, wherein determining the predicted source intensity distribution of the radiation source comprises:
determining the predicted source intensity distribution of the radiation source based on a measured transmission image.

16. The computer device according to claim 14, wherein determining the target source intensity distribution based on the calculated transmission image and the predicted source intensity distribution comprises:
correcting the predicted source intensity distribution to obtain the target source intensity distribution.

17. The computer device according to claim 16, wherein correcting the predicted source intensity distribution to obtain the target source intensity distribution comprises:
determining whether the calculated transmission image meets a preset condition, and in the case that the calculated transmission image does not meet the preset condition, correcting the predicted source intensity distribution, such that the calculated transmission image corresponding to the corrected predicted source intensity distribution meets the preset condition, and determining the corrected predicted source intensity distribution corresponding to the calculated transmission image meeting the preset condition as the target source intensity distribution.

18. The computer device according to claim 17, wherein determining the predicted source intensity distribution of the radiation source comprises determining the predicted source intensity distribution of the radiation source based on a measured transmission image, and
wherein determining whether the calculated transmission image meets the preset condition comprises:
determining a difference between the measured transmission image and the calculated transmission image; and
determining whether the calculated transmission image meets the preset condition according to the difference.

19. The computer device according to claim 17, wherein the correction is performed by a machine learning-based correction model, and
wherein the method further comprises training the correction model, and training the correction model comprises:
training an initial correction model using predicted source intensity distribution samples to obtain the correction model.

20. A method for reconstructing a dose distribution for a region of interest, comprising:
determining a predicted source intensity distribution of a radiation source;
determining a calculated transmission dose distribution based on the predicted source intensity distribution;
determining a target source intensity distribution based on the calculated transmission dose distribution and the predicted source intensity distribution; and
reconstructing the dose distribution of the region of interest based on the target source intensity distribution,
wherein determining the target source intensity distribution based on the calculated transmission dose distribution and the predicted source intensity distribution comprises:
determining whether the calculated transmission image meets a preset condition, and in the case that the calculated transmission image does not meet the preset condition, correcting the predicted source intensity distribution, such that the calculated transmission image corresponding to the corrected predicted source intensity distribution meets the preset condition, and determining the corrected predicted source intensity distribution corresponding to the calculated transmission image meeting the preset condition as the target source intensity distribution.
